(19) United States
(12) Patent Application Publication
(10) Pub. No.: US 2006/0079780 A1
(43) Pub. Date: Apr. 13, 2006

(54) Ultrasonic imaging apparatus

(75) Inventor: Hiroyuki Karasawa, Kaisei-machi (JP)

Correspondence Address: SUGHRUE MION, PLLC, 2100 PENNSYLVANIA AVENUE, N.W., SUITE 800, WASHINGTON, DC, 20037, US

(73) Assignee: FUJI PHOTO FILM CO., LTD.

(21) Appl. No.: 11/235,119

(22) Filed: Sep. 27, 2005

(30) Foreign Application Priority Data
Sep. 29, 2004 (JP) 2004-283326

(51) Int. Cl. A61B 8/06

(52) U.S. Cl. 600/447

(57) ABSTRACT

An ultrasonic imaging apparatus capable of generating ultrasonic images including boundaries between different tissues and regions divided by the boundaries in which property of boundaries and the respective tissues can be distinctly identified. The ultrasonic imaging apparatus includes ultrasonic transducers for transmitting and receiving ultrasonic waves to output reception signals; a boundary information generating unit for generating information representing positions of boundaries based on the reception signals; a first image data generating unit for generating first image data representing property of a first region and/or a second region divided by the boundaries; a second image data generating unit for generating second image data representing property of boundaries based on the reception signals; and a tissue property image data generating unit for generating tissue property image data by locating images represented by the first and second image data in the region, based on the boundary position information.

### BACKGROUND OF THE INVENTION

1. Field of the Invention

The present invention relates to an ultrasonic imaging apparatus for transmitting and receiving ultrasonic waves to perform imaging of organs, bones, etc. within a living body thereby generating ultrasonic images to be used for diagnosis.

2. Description of a Related Art

In an ultrasonic imaging apparatus to be used for medical diagnoses, an ultrasonic probe including plural ultrasonic transducers having transmitting and receiving functions of ultrasonic waves is used. When an ultrasonic beam formed by synthesizing plural ultrasonic waves is transmitted from such an ultrasonic probe to an object to be inspected, the ultrasonic beam is reflected at a boundary between regions having different acoustic impedances, i.e., between different tissues from each other within the object. Thus generated ultrasonic echoes are received and an image is constructed based on the intensity of the ultrasonic echoes, and thereby, the state within the object can be reproduced on a screen.

Recent years, when an ultrasonic image is generated, the use of elements other than intensity of ultrasonic echoes has been studied. It is conceivable that plural frequency components in the ultrasonic echo signals and statistical property (statistics values) that represents interrelationships among plural ultrasonic echo signals are utilized as the elements.

By the way, a general ultrasonic image represents shapes of tissues within the object. Accordingly, it is extremely difficult to determine tissue property of a tumor or the like, or visually recognize soft tissues separately from hard tissues in a region like the vicinity of a bone part where soft tissues such as muscles and hard tissues such as bones, tendons, and nucleus pulposus are intricate. Therefore, an ultrasonic imaging apparatus capable of imaging not only positions of boundaries between tissues but also tissue property etc. by employing ultrasonic waves is desired.

However, for example, tissue property on the internal region can be represented by utilizing statistics values such as a Rayleigh distribution, but spatial resolving power becomes lower because the calculation precision becomes lower when the range of reception signals (calculation range) used for statistics value calculation is narrow. On the other hand, position information on boundaries can be obtained by phase matching plural reception signals, but appropriate image display can not be performed with respect to a region having an indistinctive outline like a uniform internal tissue. Thus, in a conventional ultrasonic imaging apparatus, it is difficult to distinctively image tissue property of an entire range that includes both the boundaries and the internal region.

As a related technology, International Publication WO00/40997 discloses that the obtained echo signals are processed along both processing paths of one reception signal processing path using time delays set for a traditional coherent receive beam forming and another reception signal processing path using time delays set to apply incoherent summing using time delays equal to, for example, zero and an ultrasonic image is generated based on thus obtained coherent summation signals and incoherent summation signals in order to prevent incoherent summation of phase matching signals due to variations in propagation times and image deterioration in an ultrasonic image by suppressing a display based on incoherent summation signals (page 1). Further, in WO00/40997, an image is generated based on a coherence factor, and displayed as a color map overlaid on a B-mode image. Here, the coherence factor refers to the degree of similarity of a signal that has been phase matched (coherent summed signal A) and a signal that has not been phase matched (incoherent summed signal B), and expressed by the difference between the signal A and signal B, the ratio of the signal A to the signal B, or the like.

According to WO00/40997, it can be expected that the image quality of an ultrasonic image may be improved by superimposing the image obtained based on the coherence factor upon the B-mode image. However, the qualitative difference between reflector tissues is not separated into boundaries and regions divided by the boundaries to be displayed on an image.

Japanese Patent Application Publication JP-A-8-117225 discloses a living tissue evaluation apparatus including transmitting means for transmitting ultrasonic waves to a living tissue, intensity distribution obtaining means for obtaining an intensity distribution of ultrasonic waves by receiving ultrasonic waves that have been transmitted through the living tissue and spread, and evaluation value computing means for calculating an evaluation value of the living tissue based on the obtained intensity distribution for analyzing a microscopic structure of the living body by utilizing information on spatial spreading of ultrasonic waves transmitted through the living tissue (page 1).

However, in JP-A-8-117225, since an interference phenomenon in transmission is used, information on the depth direction of the ultrasonic beam can not be obtained and the property within the tissue are obtained only as integration information. Further, any information can not be obtained within objects except for an object within which ultrasonic interference occurs. Furthermore, although an intensity distribution is obtained among plural reception signals obtained by plural ultrasonic vibrators and the living tissue is evaluated based on the intensity distribution, boundaries between different tissues are not detected.

Further, JP-P2003-61964A discloses an ultrasonic diagnostic apparatus for applying ultrasonic pulses to an object to be inspected to obtain a tomographic image, smoothing the image by utilizing statistical property (difference from a Rayleigh distribution) of a speckle pattern, and extracting a microstructure in order to observe a minute abnormal lesion within a homogeneous tissue structure (page 2). The ultrasonic diagnostic apparatus includes analysis computation means for extracting a specific signal by using intensity or statistical property of amplitude information of echo signals generated from a part of the object, and display means for displaying a result extracted from the analysis computation means.

However, an object of JP-P2003-61964A is to leave structures other than speckles by utilizing statistical property of signals representing a speckle pattern. Accordingly, tissue property determined based on the statistical property is superimposed upon a B-mode image and displayed, but what is imaged thereby is only the tissue property within boundaries, and imaging of boundary property or making differences between boundaries and regions divided by the boundaries clearer is not performed.

JP-P2000-5180A discloses an acoustic impedance measurement apparatus for inputting trapezoidal or rectangular pulse signals from a pulser circuit to ultrasonic transducers, delaying the generated ultrasonic waves by an acoustic delaying medium, extracting parameters from frequency characteristics of reply signals generated due to ultrasonic waves returned to the ultrasonic transducers, measuring an acoustic impedance by utilizing a parameter strongly correlated with acoustic impedance, and displaying an image (page 1). Further, in the acoustic impedance measurement apparatus, an acoustic impedance is also obtained in the similar manner by extracting ultrasonic echoes on the backside of an object to be measured.

JP-P2001-170046A discloses, while citing the above JP-P2000-5180A as a conventional example, a living tissue property diagnostic apparatus including signal analysis means for receiving ultrasonic pulses reflected or transmitted within a living body, converting them into electric signals, and diagnosing tissue property of the living body from characteristic amounts of the electric signals for accurate diagnoses regardless of an object to be measured (page 1). The signal analysis means includes pulse width setting means for setting a signal pulse width of electric signals, region extracting means for extracting plural signal regions different at least in part of regions from the set signal pulse width, waveform characteristic amount calculating means for calculating predetermined characteristic amounts in each of the extracted regions, difference computing means for computing differences between calculated waveform characteristic amounts, and relating time determining means for relating results by the difference computing means with positions of living tissues that have generated the reception ultrasonic pulses by associating the results by the difference computing means with reception times of the ultrasonic pulses. In the living tissue property diagnostic apparatus, when waveforms are extracted, determination is performed by using peak values. Further, as examples of the above characteristic amounts, peak value, center frequency, relative bandwidth, 6 dB-decreased frequency primary moment, and secondary moment are cited (page 8).

In the living tissue property diagnostic apparatus, although tissue information between boundaries is displayed by differences with respect to the depth direction of frequency information of boundaries, there is no description about detection of boundary property.

### SUMMARY OF THE INVENTION

The present invention has been achieved in view of the above-mentioned problems. A first object of the present invention is to generate an ultrasonic image including boundaries between plural different tissues and regions divided by the boundaries, in which property of the boundaries and the respective tissues are distinctively identified. Further, a second object of the present invention is to understandably demonstrate tissue property of a reflector by separating ultrasonic echo signals representing the reflector and ultrasonic echo signals representing speckle components. Furthermore, a third object of the present invention is to perform image display suitable for medical diagnoses by clearly and distinctively displaying hard tissues such as bones and tendons and soft tissues.

In order to solve the above-mentioned problems, an ultrasonic imaging apparatus according to an aspect of the present invention includes: an ultrasonic probe including plural ultrasonic transducers for transmitting ultrasonic waves toward an object to be inspected and receiving ultrasonic waves reflected from the object to output plural reception signals; boundary information generating means for generating information representing positions of boundaries between plural different tissues based on the plural reception signals respectively output from the plural ultrasonic transducers; first image data generating means for generating image data representing property of a first region and/or a second region divided by the boundaries, based on the plural reception signals; second image data generating means for generating image data representing property of boundaries based on the plural reception signals; and tissue property image data generating means for generating image data representing tissue property with respect to a region within the object by locating an image represented by the image data generated by the first image data generating means and an image represented by the image data generated by the second image data generating means in the region, based on the information representing positions of boundaries.

According to the present invention, in boundaries between different tissues and a first region and/or a second region divided by the boundaries, images generated by processing using appropriate algorithms according to the respective regions are arranged, and therefore, not only shapes of the reflectors but also surface property of the reflectors, property of divided regions of the reflectors and soon can be distinctively imaged. As a result, the quality and efficiency of medical diagnoses employing ultrasonic images can be improved.

### BRIEF DESCRIPTION OF THE DRAWINGS

FIG. 1 is a block diagram showing a constitution of an ultrasonic imaging apparatus according to the first embodiment of the present invention;

FIG. 2 is a diagram for explanation of an operation of a data processing system for generating frequency images;

FIGS. 3A to 3C show an intensity distribution of reception signals when ultrasonic waves are transmitted toward a specular reflector and received;

FIGS. 4A to 4C show an intensity distribution of reception signals when ultrasonic waves are transmitted toward a scattering reflector and received;

FIGS. 5A to 5C show an intensity distribution of reception signals when ultrasonic waves are transmitted toward a inclined specular reflector and received;

FIG. 6 shows a spatial intensity distribution of reception signals;

FIG. 7 is a schematic diagram showing a synthesized image of a B-mode image and a tissue property image;

FIG. 8 is a block diagram showing a constitution of an ultrasonic imaging apparatus according to the second embodiment of the present invention;

FIG. 9 is a block diagram showing a constitution of an ultrasonic imaging apparatus according to the third embodiment of the present invention;

FIG. 10 is a diagram for explanation of an operation of a boundary correction unit shown in FIG. 9;

FIG. 11 is a block diagram showing a constitution of an ultrasonic imaging apparatus according to the fourth embodiment of the present invention;

FIG. 12 is a block diagram showing a constitution of an ultrasonic imaging apparatus according to the fifth embodiment of the present invention;

FIG. 13 is a flowchart showing an operation of a histogram analysis unit and a surface property image data generating unit according to a first example;

FIGS. 14A and 14B show a spatial intensity distribution of reception signals and a histogram created based thereon;

FIG. 15 is a chart showing classified parameters of a beta distribution;

FIGS. 16A to 16C show the cases where beta distributions become U-shaped;

FIGS. 17A to 17D show the cases where beta distributions become J-shaped;

FIGS. 18A to 18C show the cases where beta distributions become single-peaked;

FIG. 19 is a diagram for explanation of an operation of a histogram analysis unit and a surface property image data generating unit according to a third example; and

FIG. 20 is a block diagram showing a constitution of an ultrasonic imaging apparatus according to the sixth embodiment of the present invention.

### DESCRIPTION OF THE PREFERRED EMBODIMENTS

Hereinafter, preferred embodiments of the present invention will be described in detail by referring to the drawings. The same reference numbers are assigned to the same component elements and the description thereof will be omitted.

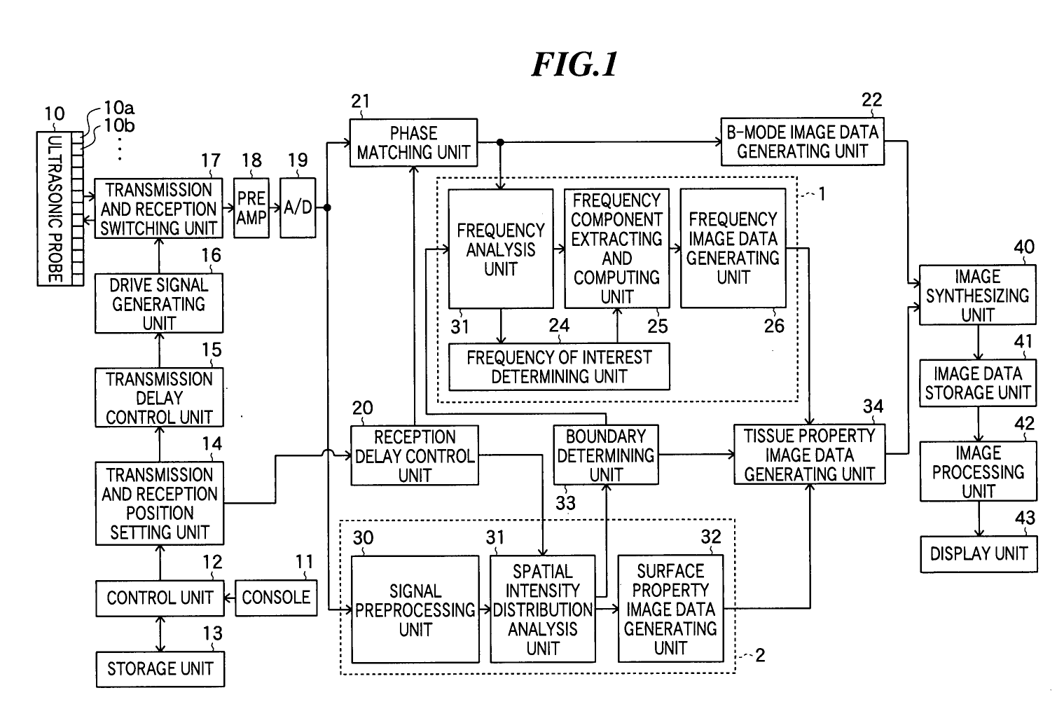

FIG. 1 is a block diagram showing a constitution of an ultrasonic imaging apparatus according to the first embodiment of the present invention. The ultrasonic imaging apparatus according to the embodiment includes an ultrasonic probe 10, a console 11, a control unit 12, a storage unit 13, a transmission and reception position setting unit 14, a transmission delay control unit 15, a drive signal generating unit 16, a transmission and reception switching unit 17, a preamplifier (PREAMP) 18, and an A/D converter 19.

The ultrasonic probe 10 is used by being abutted on the object to transmit ultrasonic waves to an object to be inspected and receive ultrasonic waves reflected from the object. The ultrasonic probe 10 includes plural ultrasonic transducers 10a, 10b, . . . for transmitting ultrasonic beams based on applied drive signals and receiving propagating ultrasonic echoes to output reception signals. These ultrasonic transducers 10a, 10b, . . . are arranged in a one-dimensional or two-dimensional manner to form a transducer array.

Each ultrasonic transducer is constituted by a vibrator in which electrodes are formed on both ends of a material having a piezoelectric property (piezoelectric material) such as a piezoelectric ceramic represented by PZT (Pb (lead) zirconate titanate), a polymeric piezoelectric material represented by PVDF (polyvinylidene difluoride), or the like. When a voltage is applied to the electrodes of the vibrator by transmitting pulse electric signals or continuous wave electric signals, the piezoelectric material expands and contracts. By the expansion and contraction, pulse ultrasonic waves or continuous wave ultrasonic waves are generated from the respective vibrators, and an ultrasonic beam is formed by synthesizing these ultrasonic waves. Further, the respective vibrators expand and contract by receiving propagating ultrasonic waves to generate electric signals. These electric signals are output as reception signals of ultrasonic waves.

Alternatively, as the ultrasonic transducers, plural kinds of elements of different conversion types may be used. For example, the above-mentioned vibrators are used as elements for transmitting ultrasonic waves and photo-detection type ultrasonic transducers are used as elements for receiving ultrasonic waves. The photo-detection type ultrasonic transducer is for detecting ultrasonic waves by converting ultrasonic signals into optical signals, and constituted by a Fabry-Perot resonator or fiber Bragg grating, for example.

The console 11 is used when an operator inputs commands and information to the ultrasonic imaging apparatus. The console 11 includes a keyboard, adjustment knob, and a pointing device including a mouse, or the like.

The control unit 12 is formed by a CPU and software, for example, and controls the respective units of the ultrasonic imaging apparatus based on the commands and information input from the console 11. In the storage unit 13, programs for allowing the CPU that forms the control unit 12 to execute operation or the like are stored.

The transmission and reception position setting unit 14 sets the transmission direction, reception direction, and depth of focus of the ultrasonic beam transmitted from the ultrasonic probe 10 and the aperture diameter of the ultrasonic transducer array (i.e., plural ultrasonic transducers to be used) in order to scan a predetermined region within the object by the ultrasonic beam. Further, the transmission delay control unit 15 sets delay times to be provided to the plural ultrasonic transducers for transmitting the ultrasonic beam that has been set by the transmission and reception position setting unit 14.

The drive signal generating unit 16 includes plural drive circuits for generating plural drive signals to be supplied to the plural ultrasonic transducers, respectively. These drive circuits generates drive signals based on the delay times that have been set in the transmission delay control unit 15.

The transmission and reception switching unit 17 switches between a transmission mode in which drive signals are supplied to the ultrasonic probe 10 and a reception mode in which reception signals are output from the ultrasonic probe 10 under the control of the control unit 11.

The preamplifier 18 and the A/D converter 19 have plural channels corresponding to the plural ultrasonic transducers 10a, 10b, . . . , input reception signals output from the plural ultrasonic transducers and perform preamplification and analog/digital conversion on the respective reception signals.

Further, the ultrasonic imaging apparatus includes a reception delay control unit 20, a phase matching unit 21, a B-mode image data generating unit 22, first image data generating means 1, second image data generating means 2, a boundary determining unit 33, a tissue property image data generating unit 34, an image synthesizing unit 40, an image data storage unit 41, an image processing unit 42, and a display unit 43.

The reception delay control unit 20 has plural delay patterns (phase matching patterns) corresponding to the reception direction and focal depth of the ultrasonic echoes, and selects delay patterns to be provided to the plural reception signals according to the reception direction and focal depth that have been set by the transmission and reception position setting unit 14 and supplies them to the phase matching unit 21 and a spatial intensity distribution analysis unit 31.

The phase matching unit 21 performs reception focus processing by providing delays to the plural reception signals (reception data) that have been A/D converted, respectively, based on the delay pattern that has been supplied from the reception delay control unit 22, and adding the signals. By the reception focus processing, sound ray signals (sound ray data) in which focal points of ultrasonic echoes are narrowed are formed.

The B-mode image data generating unit 22 generates B-mode image data by performing envelope detection processing and STC (sensitivity time gain control) on the sound ray data that has been formed in the phase matching unit 21.

The first image data generating means 1 is a data processing system for generating frequency images, and includes a frequency analysis unit 23, a frequency of interest determining unit 24, a frequency component extracting and computing unit 25, and a frequency image data generating unit 26. Here, a frequency image is formed by converting plural frequency components contained in the sound ray data into image information, and corresponds to an image that expresses tissue characteristics in a region of interest. In the embodiment, the first image data generating means 1 is provided for generating images representing property of a first region and/or a second region divided by boundaries.

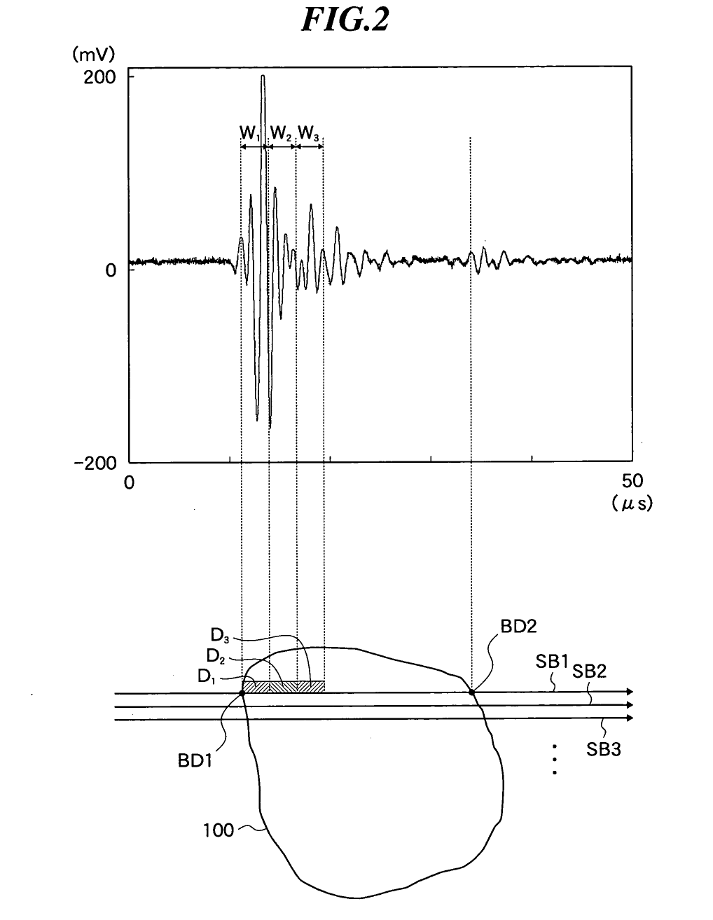

FIG. 2 is a diagram for explanation of an operation of the data processing system for generating frequency images (first image data generating means 1). In FIG. 2, there are shown a certain reflector 100 within an object to be inspected, sound rays SB1, SB2, . . . , and a waveform of sound ray signals corresponding to the sound ray SB1.

The frequency analysis unit 23 accumulates sound ray data sequentially generated in the phase matching unit 21, and performing Fourier transform on the waveform represented by the sound ray data in a predetermined range (calculation window) along a time axis (FIG. 2) to obtains plural frequency components.

Generally, frequency characteristics in sound ray data largely differ between a first region (internal region) and a second region (external region) divided by the reflector 100. Accordingly, as shown in FIG. 2, calculation windows W1, W2, . . . are desirably set so as not to cross over boundaries BD1 or BD2. Therefore, in the embodiment, the frequency analysis unit 23 sets calculation windows based on determination results of the boundary determining unit 33, which will be described later. In this regard, the widths of the calculation windows W₁, W₂, W₃, . . . may be fixed or varied according to regions. For example, since changes in frequency characteristics are great near boundaries, calculation window widths are narrowed for placing priority on spatial resolving power. Contrary, since changes in frequency characteristics are small in an internal region of a tissue such as a liver, calculation window widths are broadened for placing priority on calculation precision.

In the case where fast Fourier transform is performed in the frequency analysis unit 23, an interpolation processing unit for performing interpolation so that the number of data forming sound ray data is 2^{N} (N is an integer number) is provided in the previous stage of the frequency analysis unit 23. Further, in the case where the number of calculation units are changed with respect to each region, GUI (graphic user interface) for setting ROI (region of interest) is used.

The frequency of interest automatically determining unit 24 automatically determines a frequency of interest among plural frequencies calculated in the frequency analysis unit 23. In this regard, the frequency of interest automatically determining unit 24 may automatically determine plural frequencies which have been previously selected. Alternatively, the frequency of interest automatically determining unit 24 may automatically determine at least one frequency having a large peak or dip in a region of the whole or a part in the depth direction of the object, or use a combination of frequency components which are away from one another by a predetermined distance. Furthermore, the frequency of interest automatically determining unit 24 may use an average value of frequency components in all sound ray data or a frequency component most frequently detected, or determine one or more frequency component based on sound ray data on the direction of zero degree (front direction of the ultrasonic transducers).

The frequency component extracting and computing unit 25 extracts frequency components to be used for displaying a frequency image from the plural frequency components calculated by the frequency analysis unit 23, and further, performs predetermined computation processing by using the extracted frequency components. Thereby, characteristic amounts on the waveform in the respective calculation windows of the sound ray data are obtained. For example, one frequency component having high intensity may be extracted from the plural frequency components and output, or plural frequency components may be extracted, or a relative relationship in intensity such as a difference or ratio between the plural frequency components may be calculated and output. In the case where frequency components are determined based on frequency characteristics of a specific tissue in a region where ultrasonic echo intensity is high, the specific tissue can be displayed with more emphasis. On the other hand, in the case where frequency components are determined by focusing attention on a region where echo intensity is low, speckle components resulting from addition and interference of a large number of weak echoes may be reduced. In either case, SN ratio of each frequency component can be improved. Further, in the case where a relative value of plural frequency components is calculated, a two-dimensional distribution of a specific tissue can be obtained accurately based on the relative value.

The frequency image data generating unit 26 generates frequency image data based on the characteristic amounts output from the frequency component extracting and computing unit 25. In this regard, as shown in FIG. 2, data for display regions in different brightness or colors according to output values from the frequency component extracting and computing unit 25 are assigned to the display regions D₁, D₂, D₃, which correspond to the respective calculation windows W₁, W₂, W₃, . . . , on a frequency image.

On the other hand, the second image data generating means 2 is a data processing system for generating surface property images, and includes a signal preprocessing unit 30, the spatial intensity distribution analysis unit 31, and a surface property image data generating unit 32. Here, a surface property image refers to an image representing property of boundaries between different tissues from each other.

The signal preprocessing unit 30 performs the following intensity corrections (i) to (iii) according to need on the plural reception signals that have been A/D converted.

(i) Element Sensitivity Correction

Variations in performance of ultrasonic transducers generated when an ultrasonic transducer array is manufactured are corrected. The correction can be performed in the manner in which a correction table is created in advance by transmitting and receiving ultrasonic beams from the ultrasonic probe 10 using a standard reflection source and measuring the characteristics of the respective ultrasonic transducers, and the correction table is used at the time of processing of reception signals.

(ii) Solid Angle Intensity Correction

In an ultrasonic transducer array, since the solid angle relative to the reflection position of the ultrasonic echo becomes smaller, as an ultrasonic transducer is located closer to the end of the aperture, apparent reception intensity becomes smaller. Accordingly, intensity correction is performed on the reception signals according to the reception depth (the depth of the reflection position of the ultrasonic echoes), positional relationship with the respective ultrasonic transducers, and differences in reception solid angle between ultrasonic transducers determined by the aperture.

(iii) Distance Correction

The distance attenuation of the ultrasonic echoes that varies depending on the reception depth and positional relationship with the respective ultrasonic transducers are corrected. Since the amount of correction differs depending on the part to be observed, standard values according to parts to be observed may be set as default values in advance, and the operator may change the setting value while watching the displayed image.

Further, the signal preprocessing unit 30 performs processing such as smoothing and envelope detection on the corrected reception signals and converts those reception signals into digital signals. Thus, the envelope detection processing performed before data analysis for surface property image generation can suppress the influence by the noise and reduce the calculation amount in the subsequent processing. Furthermore, as described below, the generated surface property image data can be superimposed on the B-mode image data without change.

The spatial intensity distribution analysis unit 31 generates intensity distribution analysis information by obtaining a spatial intensity distribution (hereinafter, simply referred to as “intensity distribution”) of the plural reception signals on the same phase matching line among the plural reception signals processed in the signal preprocessing unit 30 and analyzing them. These plural reception signals on the same phase matching line are determined based on the delay pattern supplied from the reception delay control unit 20. Here, the intensity distribution analysis information includes statistics values of the plural reception signals, and those statistics values become parameters representing such property of a region as a target of analysis that the reflector surface (boundary) is hard (e.g., bone part, tendon, and ligament) or soft (e.g., skin and muscle).

The surface property image data generating unit 32 generates image data representing surface property of the reflector (surface property image data) based on the intensity distribution analysis information generated in the spatial intensity distribution analysis unit 31.

The boundary determining unit 33 determines whether a target region is a boundary between different tissues from each other or not based on the intensity distribution analysis information generated in the spatial intensity distribution analysis unit 31, and thereby, generates boundary position information representing the position of the boundary.

The principle of image generation in the second image data generating means 2 (data processing system for surface property image generation) and a method of boundary determination in the boundary determining unit 33 will be described later in detail.

The tissue property image data generating unit 34 generates image data representing an image in which surface property images are located at boundaries and frequency images are located in the internal region and/or the external region of the boundaries based on the surface property image data, frequency image data, and boundary position information. Hereinafter, such an image representing tissue property of the respective regions is referred to as a tissue property image. Further, the tissue property image data is converted into color signals when an ultrasonic image is displayed on the screen, and the boundaries and the internal region and/or the external region of the boundaries are displayed in different colors according to the characteristics thereof in the tissue property image.

The image synthesizing unit 40 generates synthesized image data in which a tissue property image is superimposed upon corresponding regions of the B-mode image based on the B-mode image data generated by the B-mode image generating unit 22 and the tissue property image data generated in the tissue property image data generating unit 34. The regions on the B-mode image on which the tissue property image is to be superimposed may be automatically determined by the image synthesizing unit 40, or may be manually designated by the operator using the console 11.

The image data storage unit 41 stores generated synthesized image data. Further, the image processing unit 42 generates image data for screen display by performing predetermined image processing including scan conversion, gradation processing, and the like on the synthesized image data. The display unit 43 includes a display device such as a CRT or LCD, and displays an ultrasonic image based on the image data that has been image processed in the image processing unit 42.

Next, the principle of surface property image generation will be described.

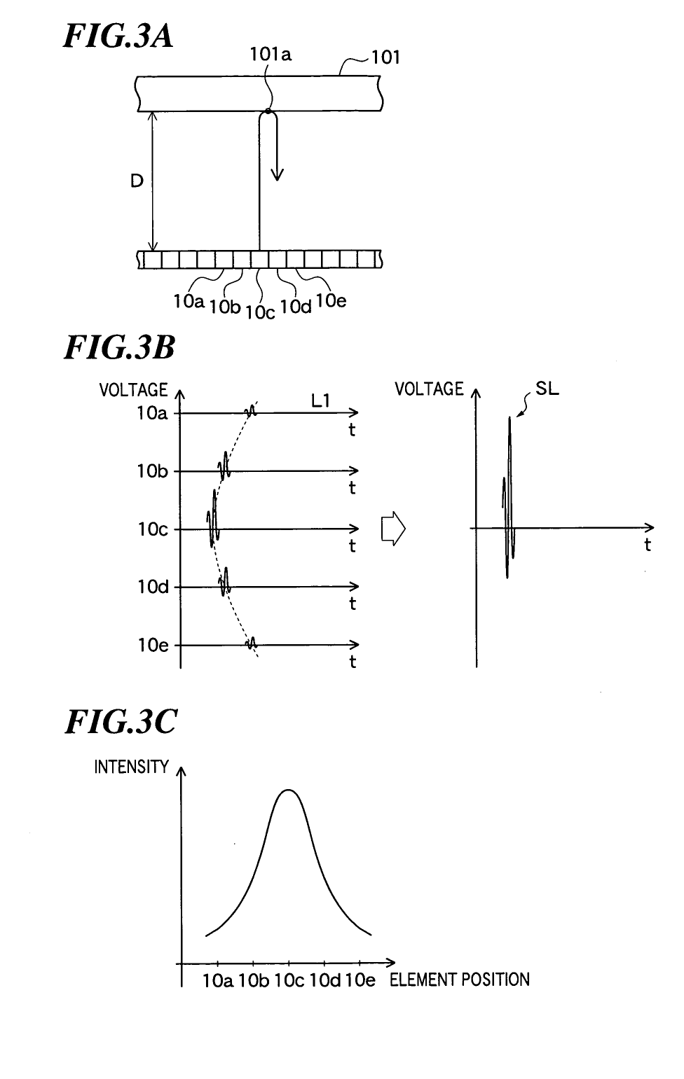

First, as shown in FIG. 3A, a case will be considered where an ultrasonic beam is transmitted toward a reflector 101 and an ultrasonic echo reflected on the surface of the reflector 101 located at depth “D” is received by using an ultrasonic transducer array including ultrasonic transducers 10a to 10e. FIG. 3B shows reception waveforms of ultrasonic echoes at the ultrasonic transducers 10a to 10e. In FIG. 3B, the horizontal axis indicates time (t) and the vertical axis indicates voltage of reception signal. Further, FIG. 3C shows an intensity distribution of the reception signals output from the ultrasonic transducers 10a to 10e. In FIG. 3C, the horizontal axis indicates position of ultrasonic transducer (element) and the vertical axis indicates intensity of reception signal.

The ultrasonic echoes reflected at reflection point 101a are first received by the ultrasonic transducer 10c right opposite to the reflection point 101a, and then, sequentially received by the ultrasonic transducers 10b and 10d and the ultrasonic transducers 10a and 10e as shown in FIG. 3B. In the case where the reflector 101 is an object that reflects the ultrasonic echoes with little scattering like a bone part, the ultrasonic echoes are received by the ultrasonic transducers 10a to 10e in an intensity distribution with the position of the ultrasonic transducer 10c as a peak thereof as shown in FIG. 3C. As below, such a reflector (reflection surface) is called “specular reflector (specular reflection surface)” and the ease of specular reflection of the reflector surface (i.e., difficulty of scattering) is called “specular reflectance”.

In the case where the B-mode image is generated, a predetermined delay times are provided to the reception signals on the same phase matching line L1 and added them. Thereby, a sound ray signal SL representing ultrasonic information on a region including the reflection point 101a is formed.

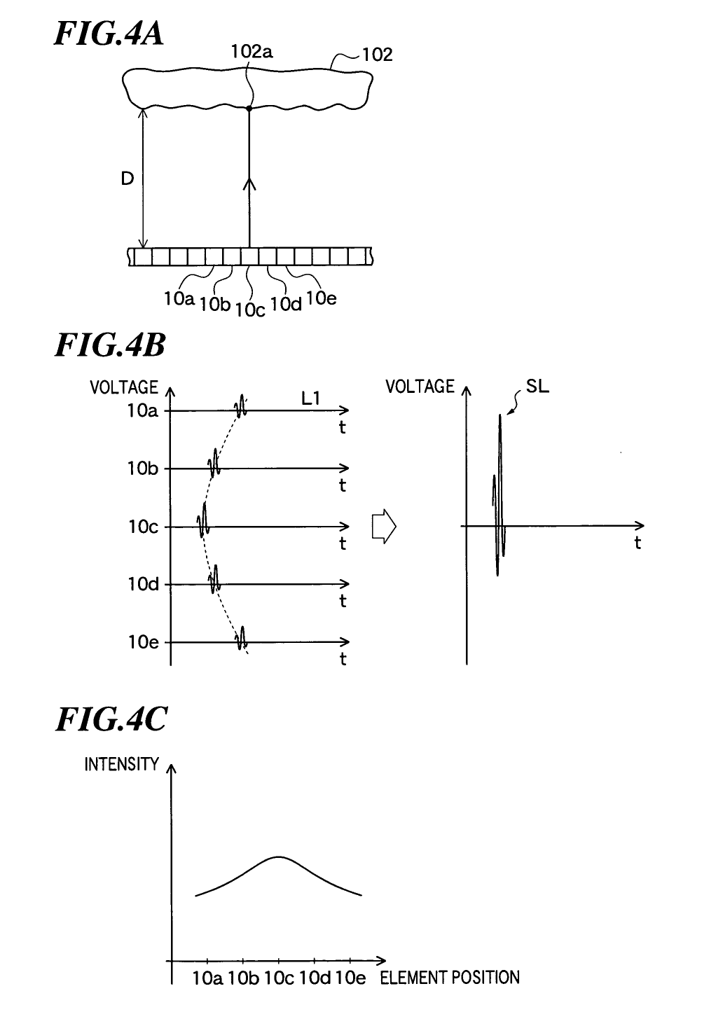

Next, the case where an ultrasonic beam is transmitted toward a reflector like a soft tissue that readily scatters ultrasonic waves will be considered. As below, such a reflector (reflection surface) is called “scattering reflector (scattering reflection surface)”. As shown in FIG. 4A, when an ultrasonic beam is transmitted toward a scattering reflector 102 located at depth “D”, the ultrasonic beam is scattered in various directions at reflection point 102a as shown in FIG. 4B. Thus generated ultrasonic echoes are received by the ultrasonic transducers 10a to 10e with timing depending on the depth “D” and the position of the reflection point 102a. Since the timing is on the phase matching line L1 like the case of the reception waveform of the ultrasonic echoes shown in FIG. 3B, when phase matching is performed for generating a B-mode image, the same sound ray signal SL as shown in FIG. 3B is formed.

However, in the case where an ultrasonic beam is reflected by the scattering reflector, because the intensity of ultrasonic echoes is dispersed in various directions, the intensity distribution of the reception signals output from the ultrasonic transducers 10a to 10e becomes relatively flat as shown in FIG. 4C.

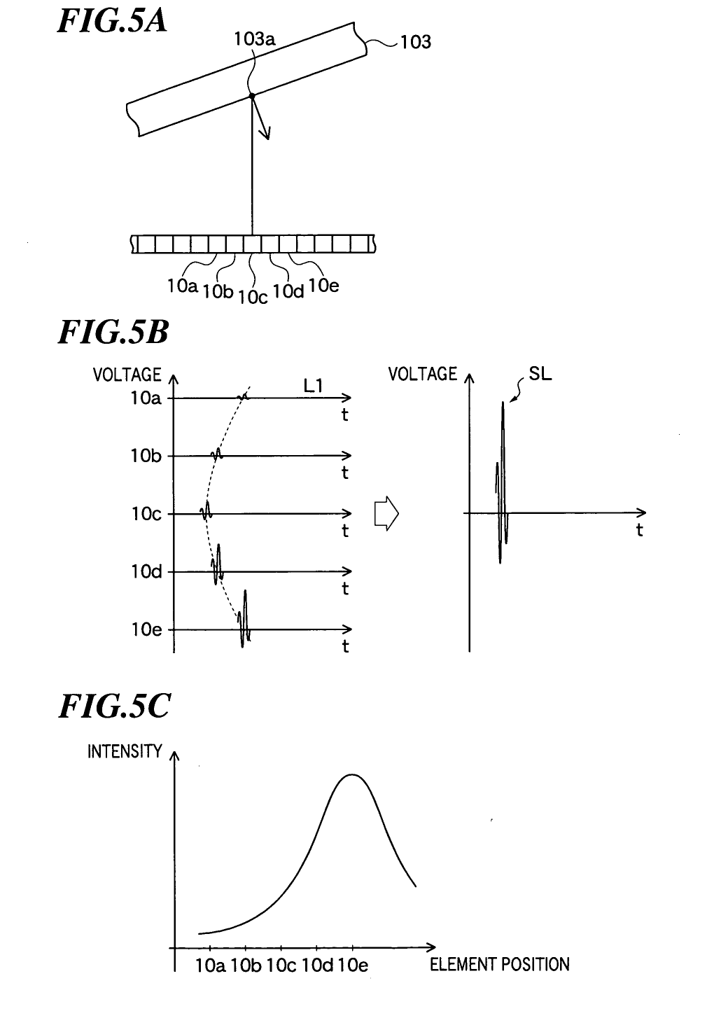

Next, the case where a specular reflector is inclined relative to the ultrasonic transducer array will be considered. As shown in FIG. 5A, when an ultrasonic beam is transmitted toward a specular reflector 103 located at depth “D”, the ultrasonic beam is reflected in a direction different from the direction in which the ultrasonic beam has been transmitted according to the inclination of the specular reflector 103. Thus generated ultrasonic echoes are received by the ultrasonic transducers 10a to 10e with timing depending on the depth “D” and the position of the reflection point 103a. As shown in FIG. 5B, since the timing is on the phase matching line L1 like the case of the reception waveform of ultrasonic echoes shown in FIG. 3B, when phase matching is performed for generating a B-mode image, also the same sound ray signal SL as shown in FIG. 3B is formed.

However, in the case where the ultrasonic beam is reflected by the reflector inclined relative to the ultrasonic transducer array, since the propagation direction of ultrasonic echoes is changed, the peak is shifted in the intensity distribution of the reception signals output from the ultrasonic transducers 10a to 10e as shown in FIG. 5C.

Thus, when phase matching is performed on the reception signals, the sound ray signals representing the reflection positions (the boundary positions) of the ultrasonic echoes are uniformly determined, and the surface condition and inclination of the reflector can be obtained by focusing attention on the interrelationship among plural reception signals (e.g., intensity distribution). Especially, the reflectance of a bone part becomes about hundred times the reflectance of a soft tissue, and therefore, it can be analyzed at the respective reception signal levels and the surface condition of the reflector can be sufficiently discriminated.

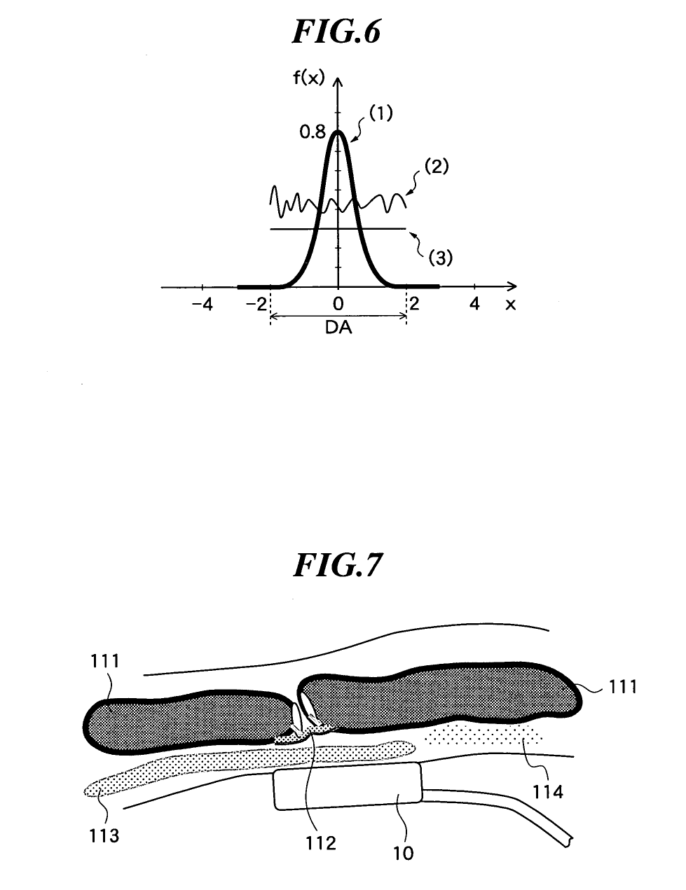

Next, a method of imaging the tissue property based on the interrelationship among plural reception signals will be described by referring to FIG. 6.

First, the spatial intensity distribution analysis unit 31 shown in FIG. 1 obtains an intensity distribution of plural reception signals with respect to a region as a target of analysis (analysis region). That is, in a graph having the horizontal axis as position coordinate of transducer and the vertical axis as intensity of reception signal, intensity of the reception signals on the same phase matching line output from the plural ultrasonic transducers within aperture diameter DA of the ultrasonic transducers is plotted. Then, in the intensity distribution chart, the horizontal axis is read as data value and the vertical axis is read as frequency from a different perspective. As shown in FIG. 6, thus obtained relationship diagram is handled as a frequency distribution chart representing the relationship between random probability “x” and probability density function f(x) as below.

In FIG. 6, curve (1) represents a frequency distribution in the case where the frequency distribution is concentrated on a certain value, that is, an ultrasonic beam is reflected by a specular reflector. Further, curve (2) represents a frequency distribution in the case where the frequency is randomly distributed, that is, an ultrasonic beam is reflected by a scattering reflector. Furthermore, curve (3) shown for comparison represents a frequency distribution in the virtual case where an ultrasonic beam is reflected in plural directions with equal intensity.

The spatial intensity distribution analysis unit 31 calculates the following parameters (1) to (4) based on the frequency distributions.

(1) Mean

A mean is used as a value representing quantitative characteristics of frequency. When an ultrasonic echo propagating from the front direction of the ultrasonic transducer array is received, the mean typically becomes zero (center), while, when a reflector is inclined relative to the ultrasonic transducer array, the mean is shifted from the center toward an end. Not only the typical arithmetic mean but also median or mode is used. Since the magnitude relationship between these arithmetic means, medians, or modes changes according to the distribution conditions of frequency, they can be used when variations in frequency are estimated.

(1-1) Median

A median refers to a value located at the center of the number of data in the case where the frequencies are arranged in order from the minimum value. When the number of data is even, the arithmetic mean of the center two values is used.

(1-2) Mode

A mode refers to a value with the highest frequency among frequencies.

(2) Variance

A variance is one of scales that indicate variations in frequency, and obtained by dividing sum of squares of deviation as differences between the respective detection data and arithmetic mean by the number of data (or the number of data−1). When the frequency distribution is close to the normal distribution and the peak rises as the curve (1), a variance value becomes smaller. Contrary, when the frequency distribution is random as the curve (2) or when the frequency distribution is uniform as the curve (3), a variance value becomes larger.

(3) Skewness

A skewness refers to a scale that indicates the degree of asymmetry around the mean of frequency, and is obtained by the following expression.

$$Skewness = ( sum of cube of deviation ) / ( number of data ) / ( cube of standard deviation )$$

Zero of skewness represents that the frequency distribution is not deviated, and in this case, the arithmetic mean, the median, and the mode become equal. Further, positive skewness represents that the frequency distribution is negatively deviated, and, in this case, the relationship arithmetic mean>median>mode holds. Furthermore, negative skewness represents that the frequency distribution is positively deviated, and in this case, the relationship arithmetic mean<median<mode holds.

(4) Kurtosis

A kurtosis refers to a scale that indicates degree of concentration around the mean of frequency (sharpness), and is obtained by the following expression.
Kurtosis=(sum of biquadrate of deviation)/(number of data)/(cube of standard deviation)

Here, in a standard normal distribution having a mean of “0” and variance of “1”, the kurtosis becomes “3”. Accordingly, the kurtosis is evaluated with numeric value “3” as a reference value. That is, when the kurtosis is “3”, the frequency distribution is close to the normal distribution. Further, the smaller than “3” the kurtosis becomes, flatter the frequency distribution becomes. Furthermore, the larger than “3” the kurtosis becomes, sharper the frequency distribution around the mean becomes.

The surface property image data generating unit 32 shown in FIG. 1 generates surface property image data by assigning predetermined colors to display regions on the ultrasonic image corresponding to the analysis region based on the parameters calculated in the spatial intensity distribution analysis unit 31. For example, a bluish color is assigned to a region where the variance is smaller than a predetermined threshold value as shown by curve (1) in FIG. 6 (reflection points of a specular reflector), and, according to the values of variance and kurtosis, density or saturation of the color assigned to the corresponding display region is changed.

Further, the boundary determining unit 33 shown in FIG. 1 determines, for example, that a region, where the variance is smaller than a predetermined threshold value, is a boundary based on the parameters calculated by the spatial intensity distribution analysis unit 31. Alternatively, the unit may determine the region where the kurtosis is larger than a predetermined threshold value is a boundary.

FIG. 7 schematically shows a synthesized image of a B-mode image and a tissue property image. In an ultrasonic image shown in FIG. 7, the boundaries represented by the surface property image data, i.e., the surfaces of a bone part 111 and a ligament 112 are displayed in different density according to specular reflectance. Further, the internal region and/or the external region of boundaries represented by the frequency image data, i.e., uniform tissues within the bone part 111, a muscle tissue 113, and a speckle region 114 are displayed in different colors. Thus, an imaging region with an object to be inspected is separated into boundaries and the internal region and/or the external region thereof, images obtained by performing appropriate data processing with respect to the respective tissues are synthesized, and thereby, an ultrasonic image with advantageous discrimination in which characteristics of boundaries and the respective regions are clearly shown. Especially, real tissues can be understandably demonstrated by separately displaying the speckle region 114. Further, in a frequency image, calculation windows are appropriately set with the surface of the reflector as a boundary, and thereby, the internal region of a lesion part such as a tumor can be imaged clearly and distinctively from the external region, and judgment as to whether the part is malignant or benign can be easily made. Furthermore, those regions are simultaneously displayed by a synthesized image, and thereby, accurate tissue information can be grasped, and the quality and efficiency of medical diagnoses can be improved. For example, even in a region like the vicinity of a bone part where soft tissues such as muscles and hard tissues such as bones, tendons, and nucleus pulposus are intricate, the respective tissues can be distinctively displayed, and thereby, the synthesized image is thought to be effective in the orthopedic field.

In the above-mentioned embodiment, different signal preprocessings have been performed in the B-mode image data generating unit 22, the image data generating means 1, and the image data generating means 2, however, a common preprocessing may be performed. For the purpose, the signal preprocessing unit 30 shown in FIG. 1 may be located before the branch to the phase matching unit 21 and the spatial intensity distribution analysis unit 31. In this case, such signal preprocessing may be performed before A/D conversion of reception signals or after the A/D conversion.

Further, in the embodiment, coefficients of signal gain and noise filter may be changed between the image data generating means 1 and the image data generating means 2. For example, in the image data generating means 1 for generating images representing property of the internal region and/or the external region of boundaries, the SN ratio can be improved by cutting high frequency components.

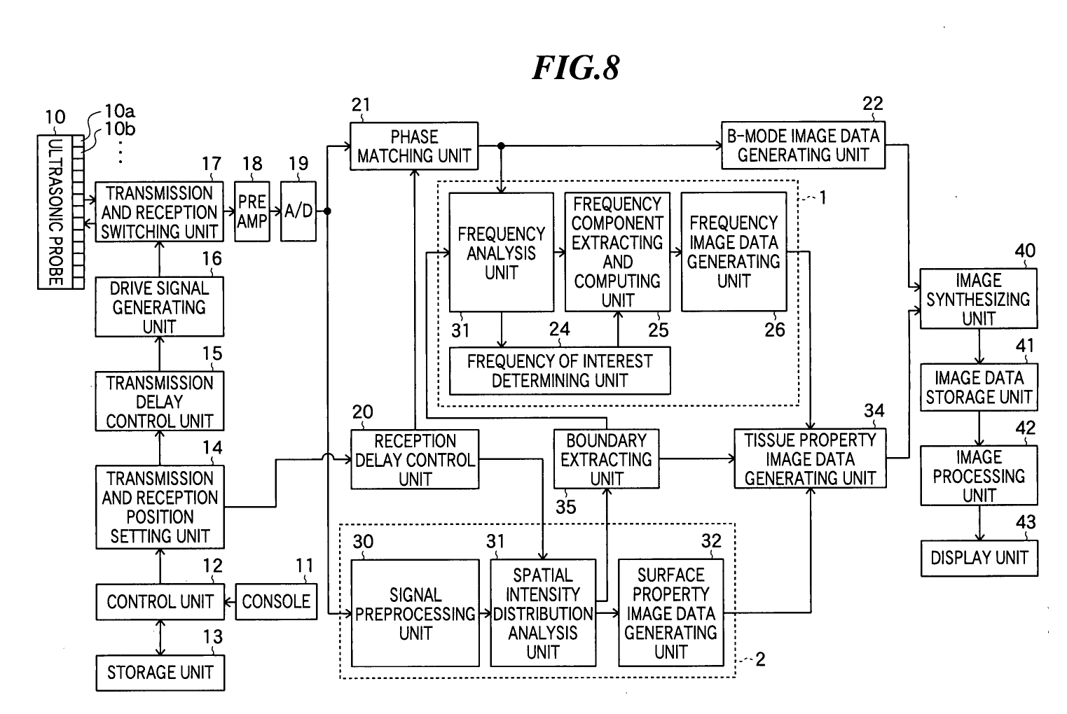

Next, an ultrasonic imaging apparatus according to the second embodiment of the present invention will be described. FIG. 8 is a block diagram showing a constitution of the ultrasonic imaging apparatus according to the embodiment.

As shown in FIG. 8, this ultrasonic imaging apparatus has a boundary extracting unit 35 in place of the boundary determining unit 33 shown in FIG. 1. Other constitution is the same as that of the ultrasonic imaging apparatus shown in FIG. 1.

The boundary extracting unit 35 generates boundary position information by extracting boundaries within the object based on the sound ray data generated in the phase matching unit 21. Here, referring to FIG. 2 again, at the boundary of the reflector, voltages of the signals representing sound rays drastically change. Accordingly, the boundary extracting unit 35 can extract positions where the voltages of the signals representing sound rays are peaked as boundaries (e.g., boundary BD2). Alternatively, in the case where, in adjacent two sound rays, the difference between peak values of voltages in corresponding positions is larger than a predetermined threshold value, the position may be extracted as a boundary (e.g., boundary BD1).

Furthermore, as a modified example of the ultrasonic imaging apparatus according to the second embodiment, the boundary extracting unit may generate boundary position information based on the sound ray data generated in the phase matching unit 21 and the information representing surface property of the reflector generated in the spatial intensity distribution analysis unit 31. For example, the specular reflectance is determined with respect to a position where a peak of waveform appears in the sound ray data based on the statistics values such as variance and kurtosis, and thereby, boundaries can be extracted more accurately without greatly increasing the amount of calculation.

In addition, in the embodiment, boundary extraction may be performed using publicly known means.

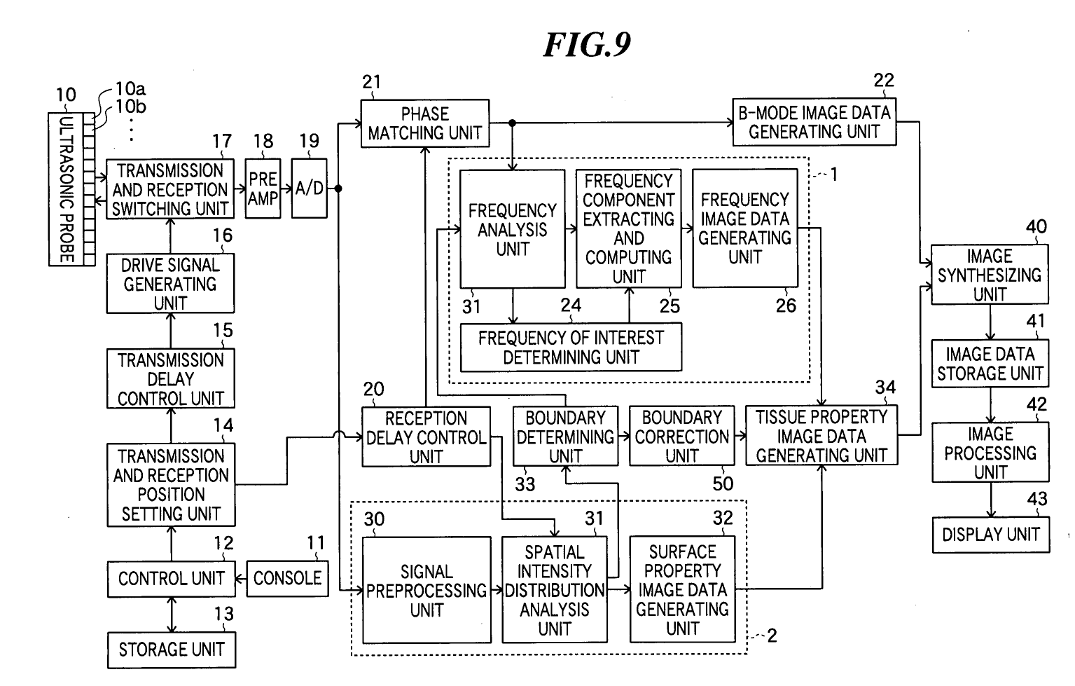

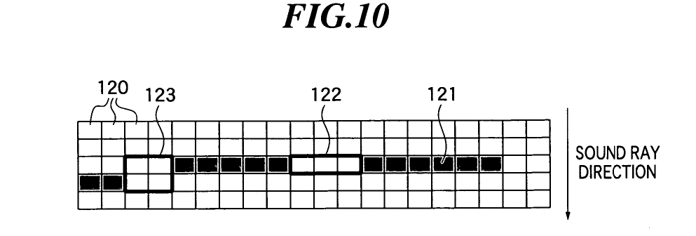

Next, an ultrasonic imaging apparatus according to the third embodiment of the present invention will be described by referring to FIGS. 9 and 10. FIG. 9 is a block diagram showing a constitution of the ultrasonic imaging apparatus according to the embodiment. As shown in FIG. 9, this ultrasonic imaging apparatus further has a boundary correction unit 50 in addition to the ultrasonic imaging apparatus shown in FIG. 1. Other constitution is the same as that of the ultrasonic imaging apparatus shown in FIG. 1.

FIG. 10 is a diagram for explanation of an operation of the boundary correction unit 50. In FIG. 10, gray regions show pixels 121 that have been determined to be boundaries by the boundary determining unit 33 of plural pixels 120 forming an ultrasonic image.

Here, as described above, the boundary determining unit 33 determines whether the respective regions on sound rays are boundaries or not. Accordingly, depending on scanning density or solving power of ultrasonic beams, like regions 122 and 123, they are not determined as boundaries even though they really are boundaries. As a result, it is likely that an unnatural image in which there is an error in arrangement of frequency images to be determined with reference to the position of the boundary, or the like might be generated.

In order to avoid such an artifact (virtual image) , when accumulating sound ray data for one screen, the boundary correction unit 50 analyzes the continuity of the boundary between adjacent pixels in the horizontal direction (a direction perpendicular to the sound ray direction) or diagonal direction, and correction is performed for regarding pixel regions as a boundary in the case where a boundary for plural pixels continues and then the boundary is disrupted for several pixels.

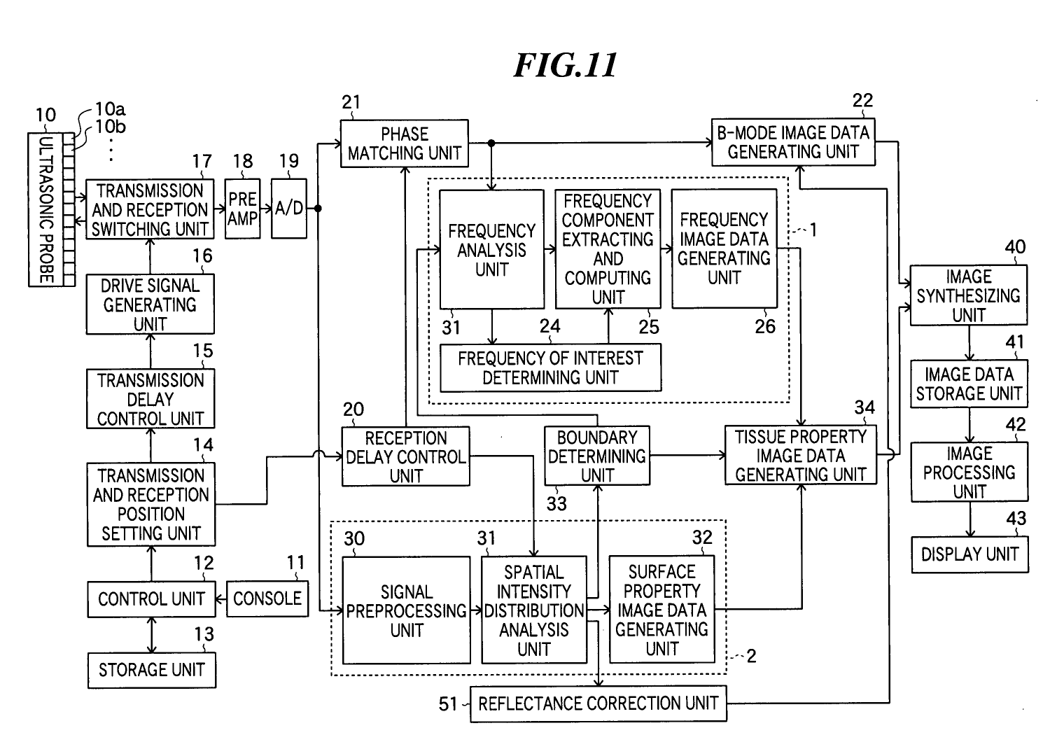

Next, an ultrasonic imaging apparatus according to the fourth embodiment of the present invention will be described. FIG. 11 is a block diagram showing a constitution of the ultrasonic imaging apparatus according to the embodiment. As shown in FIG. 11, this ultrasonic imaging apparatus further has a reflectance correction unit 51 compared to the ultrasonic imaging apparatus shown in FIG. 1. Other constitution is the same as that of the ultrasonic imaging apparatus shown in FIG. 1.

The reflectance correction unit 51 provides amounts of correction for correcting B-mode image data to the B-mode image data generating unit 22 based on the parameters calculated by the spatial intensity distribution analysis unit 31.

Here, referring FIGS. 3A and 5A again, the case where ultrasonic beams with the same intensity are transmitted to specular reflectors 101 and 103 having the same surface property will be considered. As shown in FIG. 5A, when the specular reflector 103 is inclined relative to the incident direction of the ultrasonic beam, because the ultrasonic beam is reflected in a direction different from the incident direction, the case where only part of the beam is received by the ultrasonic transducers 10a, 10b, . . . occurs. As a result, the intensity of reception signals becomes low, and thereby, despite the essentially strong specular reflector, it is only recognized as a weak diffusion distribution. Accordingly, in the embodiment, data values are corrected based on the inclination of the reflector so that B-mode image data may represent real reflectance of reflector surfaces.

The reflectance correction unit 51 has a table for reflectance correction in which amounts of correction corresponding to parameters for reflectance correction are stored, and outputs the amounts of correction corresponding to parameters on the respective analysis regions calculated by the spatial intensity distribution analysis unit 31 to the B-mode image data generating unit 22. As the parameters for reflectance correction, mode, kurtosis, or the like may be used. For example, zero of the mode represents that the reflector is not inclined as shown in FIG. 3A, and, in this case, the amount of correction of B-mode image data also becomes zero. Further, since the larger the absolute value of the mode, the larger the inclination of the reflector becomes as shown in FIG. 5A, the amount of correction of B-mode image data also becomes larger.

The table for reflectance correction can be created in the following manner, for example. That is, transmission and reception of ultrasonic beams are performed from the ultrasonic probe 10 while varying the inclination of a standard reflector, and parameters (e.g., mode) are calculated thereby obtained reception signals. On the other hand, the rate of decrease in detection intensity of ultrasonic beam generated according to the inclination of the standard reflector is obtained, and the rate of decrease may be associated with parameters as an amount of correction through the inclination of the standard reflector.

Thus, according to the embodiment, a B-mode image can be displayed based on the real reflectance, i.e., accurate difference between acoustic impedances. By the way, the inclination obtained by the parameters for reflectance correlation may be used for outline correction (interpolation) in the B-mode image. Thereby, the continuity of the outline can be improved, and an easily-viewable ultrasonic image in which shapes of reflectors are distinctively shown can be generated.

Further, the reflectance correction unit in the embodiment may be provided in the ultrasonic imaging apparatus according to the second or third embodiment of the present invention.

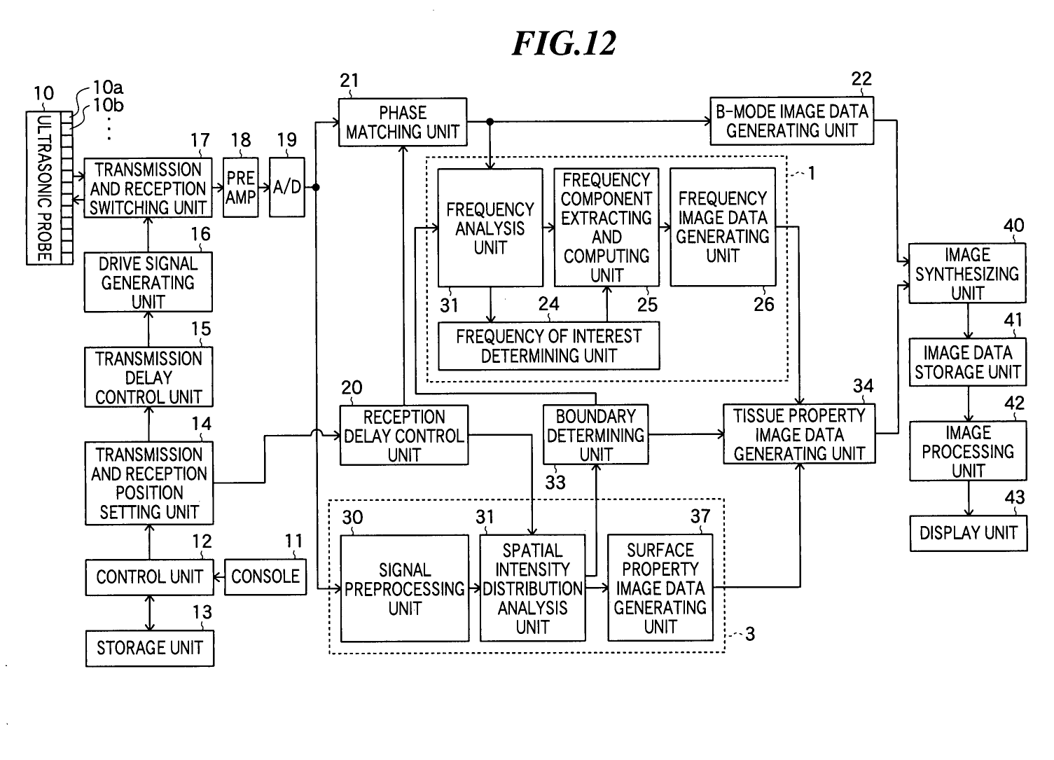

Next, an ultrasonic imaging apparatus according to the fifth embodiment of the present invention will be described. FIG. 12 is a block diagram showing a constitution of the ultrasonic imaging apparatus according to the embodiment. As shown in FIG. 12, this ultrasonic imaging apparatus has image data generating means 3 including a histogram analysis unit 36 and a surface property image data generating unit 37 in place of the image data generating means 2 including the spatial intensity distribution analysis unit 31 and the surface property image data generating unit 32 shown in FIG. 1. The image data generating means 3 is a data processing system for generating surface property images. Other constitution is the same as that of the ultrasonic imaging apparatus shown in FIG. 1.

The histogram analysis unit 36 generates histogram analysis information by creating a histogram based on plural reception signals on the same phase matching line of the plural reception signals that have been intensity corrected by the signal preprocessing unit 30 and analyzing it. The histogram analysis information includes statistics values of plural reception signals and the statistics values are used as parameters representing property of regions as a target of analysis. Further, the surface property image data generating unit 37 generates surface property image data based on the histogram analysis information generated in the histogram analysis unit 36.

As below, an operation of the histogram analysis unit 36 and the surface property image data generating unit 37 will be described in detail.

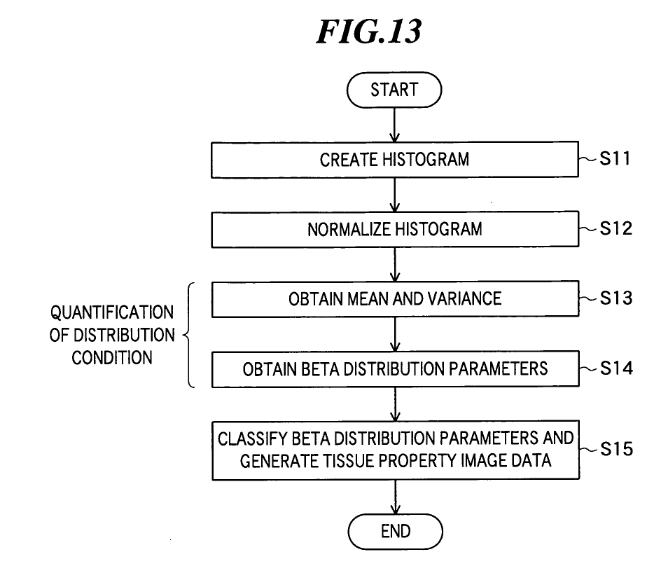

FIG. 13 is a flowchart showing an operation of the histogram analysis unit 36 and the surface property image data generating unit 37 shown in FIG. 12 according to a first example.

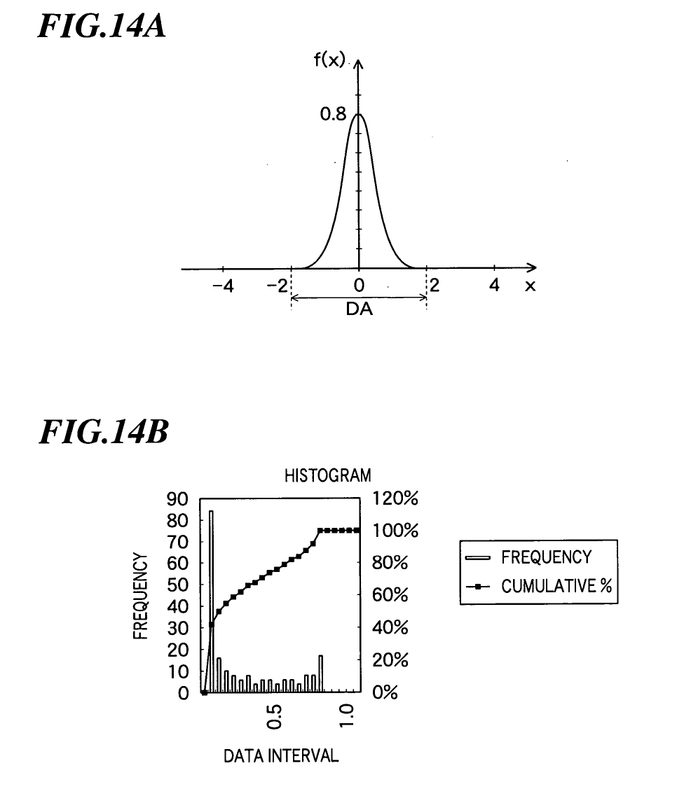

At step S11 in FIG. 13, the histogram analysis unit 36 obtains an intensity distribution as shown in FIG. 14A with respect to reception signals on a region as a target of analysis (analysis region) on a reflector, and further, creates a histogram shown in FIG. 14B based on the intensity distribution. Here, FIG. 14A shows the intensity distribution of reception signals output from plural ultrasonic transducers within aperture diameter DA of an ultrasonic transducer array.

Then, at step S12, the histogram analysis unit 36 normalizes the created histogram so that the range of values (the horizontal axis of the histogram) may be “0” to “1”.

Then, at steps S13 and S14, the histogram analysis unit 36 qualifies the distribution condition of the normalized histogram using a beta distribution. Here, The beta distribution is expressed using shape parameters α and β by X˜B(α,β), and probability density function f(x) in the beta distribution, r-th moment (product moment) about origin, mean E(x), variance VAR(x), and mode MOD are expressed by the following expressions (1) to (5).

$$\begin{matrix}f ( x ) = \frac{1}{B ( α , β )} {{x}^{α - 1} ( 1 - x )}^{β - 1} ( 0 \leq x \leq 1 ) & ( 1 ) \\ {μ}_{r} = \frac{B ( α + r , β )}{B ( α , β )} ( r \geq 1 ) & ( 2 ) \\ E ( x ) = \frac{α}{α + β} & ( 3 ) \\ VAR ( x ) \frac{α β}{{( α + β )}^{2} ( α + β + 1 )} & ( 4 ) \\ MOD = \frac{α - 1}{α + β - 2} ( α > 1 , β > 1 ) & ( 5 )\end{matrix}$$

In order to obtain the beta distribution, first, at step S13, sample mean x_{AVE} and variance σ² are obtained using the following expressions (6) and (7) from the normalized histogram.

$$\begin{matrix}{x}_{AVE} = \frac{1}{N} {\sum }_{i = 1}^{n} {f}_{i} {m}_{i} & ( 6 ) \\ {σ}^{2} = \frac{1}{N} {\sum }_{i = 1}^{n} {f}_{i} {m}_{i}^{2} - {X}_{AVE}^{2} & ( 7 )\end{matrix}$$

Then, at step S14, beta distribution parameters α and β are obtained by estimation according to a moment method using the following expressions (8) and (9).

$$\begin{matrix}α \text{:} {x}_{AVE} \{ [ {x}_{AVE} ( 1 - {x}_{AVE} ) / ( \frac{n - 1}{n} ) {σ}^{2} ] - 1 \} & ( 8 ) \\ β \text{:} ( 1 - {x}_{AVE} ) \{ [ {x}_{AVE} ( 1 - {x}_{AVE} ) / ( \frac{n - 1}{n} ) {σ}^{2} ] - 1 \} & ( 9 )\end{matrix}$$

Thereby, an approximate distribution to the beta distribution is obtained.

At step S15, as shown in FIG. 15, the surface property image data generating unit 37 generates surface property image data by classifying the beta distribution parameters and assigning predetermined colors on the display regions on the ultrasonic image corresponding to the analysis region according to the values of α and β. Here, “U-shaped”, “J-shaped”, and “single-peaked” represent shapes of the probability density function in the beta distribution.

(i) The Case where α<1 and β<1

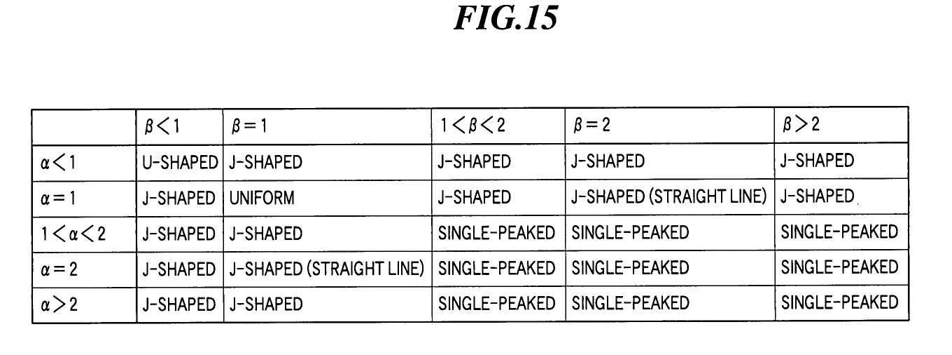

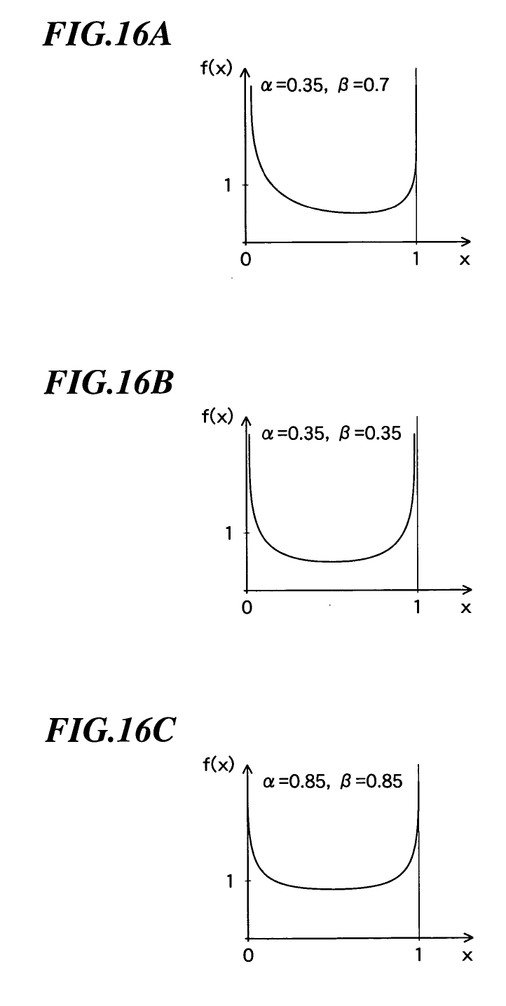

In this case, as shown in FIGS. 16A to 16C, the probability density function f(x) becomes U-shaped. The peak rises in the intensity distribution of reception signals as shown in FIG. 14A and this represents that the reflector surface is the hard tissue that specularly reflects ultrasonic waves. Accordingly, bluish colors are assigned to the tissue property image data of the display regions corresponding to the analysis region. In this regard, as shown in FIG. 16A or 16B, since the smaller the value |α×β|, the steeper the U-shaped gradient of the probability density function f(x) becomes, that represents strong specular reflection, and thereby, deep blue is assigned thereto. Contrary, as shown in FIG. 16C, since the larger the value |α×β|, the gentler the U-shaped gradient of the probability density function f(x) becomes, the specular reflection becomes weak, and thereby, pale blue is assigned thereto.

(ii) The Case where (α−1)×(β−1)≦0

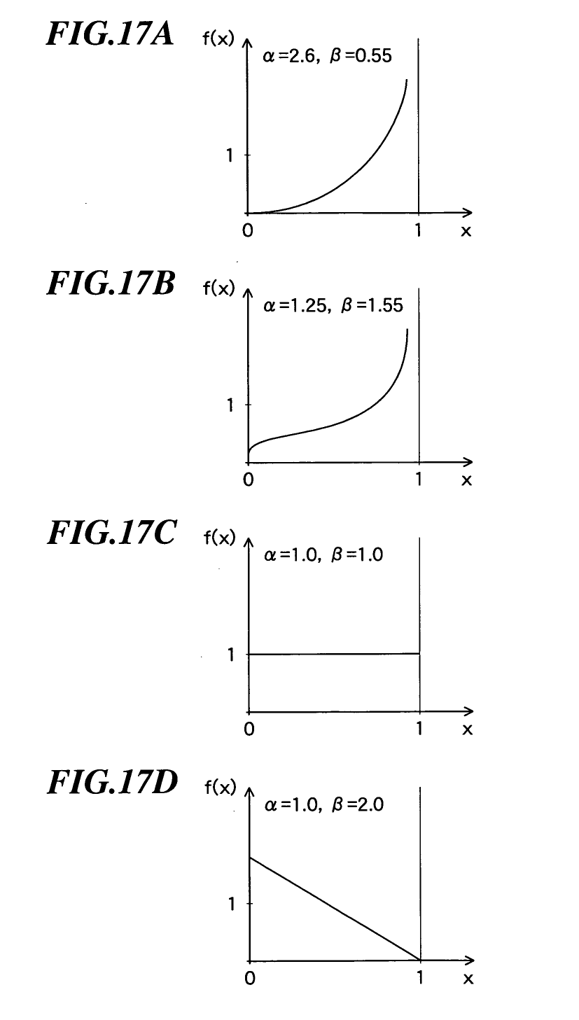

In this case, as shown in FIGS. 17A to 17D, the probability density function becomes J-shaped. The specular reflection has a peak rising to some degree in the intensity distribution of reception signals and this represents that the peak center of intensity resides outside of the aperture of the transducer array.

In this case, bluish colors may be assigned to the surface property image data of the display regions corresponding to the analysis region, or greenish colors maybe assigned thereto in order to discriminate the angle of the reflector from that in the above case (i). Further, as shown in FIG. 17A or 17B, since the more distant from “1” the value |α/β|, the steeper the gradient of the J-shape becomes, that represents strong specular reflection, and thereby, deep blue or green is assigned thereto. Contrary, as shown in FIG. 17C or 17D, since the closer to “1” the value |α/β|, the gentler the gradient of the J-shape becomes (e.g., gradient “0” ) , that represents weak specular reflection, and thereby, pale blue or green is assigned thereto.

(iii) The case where α>1 and β>1

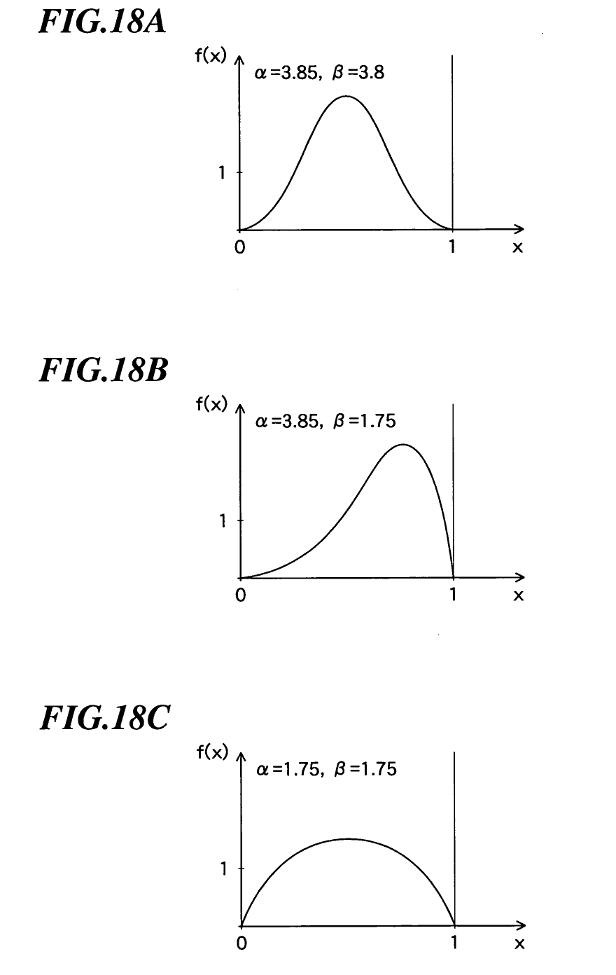

In this case, as shown in FIGS. 18A to 18C, the probability density function f(x) becomes single-peaked. That is, this represents that the intensity distribution of reception signals is a normal distribution and the analysis region is a tissue that scatter reflects ultrasonic waves.

Next, an operation of the histogram analysis unit 36 and the surface property image data generating unit 37 (FIG. 12) according to a second example will be described.

In this example, in the same manner as have been described in the first example, an intensity distribution with respect to reception signals on the analysis region is obtained and a histogram is created, and various statistics values are calculated based on a histogram obtained by normalizing that histogram. As the statistics values, mode, median, quartile deviation, skewness, frequency, etc. are used. Here, the quartile deviation is an indicator representing the degree of scattering of frequency, and the quartile deviation QR is obtained by the following expression using the first quartile X_{0.25} and the third quartile X_{0.75}. The quartile is a value in a position where the frequency is divided into quarters when data is aligned in ascending order, and the first quartile is a value located at 25% in ascending order and the third quartile is a value located at 75% in ascending order.
QR=(X_{0.75}−X_{0.25})/2
Further, other statistics values are the same as those have been described in the first embodiment.

Then, the surface property image data generating unit 37 generates surface property image data by assigning predetermined colors to display regions corresponding to the analysis regions based on the calculated statistics values. For example, in a condition in which the variation from the mean of the frequency distribution is large, the variance σ², quartile deviation, or skewness becomes large.

Accordingly, in the case where these statistics values are larger than predetermined threshold values, the analysis region is regarded as a boundary, and different bluish colors are assigned to the surface property image data of the corresponding display regions according to the values. In this case, the beta distribution becomes U-shaped or J-shaped. In this regard, as the curve (3) in FIG. 6, the respective statistics values when the frequency has a uniform distribution may be used as threshold values.

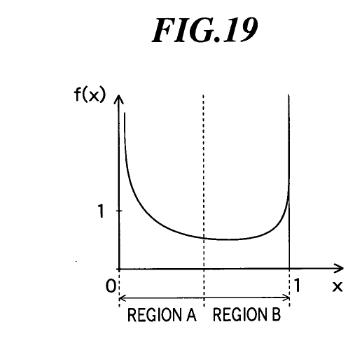

Next, an operation of the histogram analysis unit 36 and the surface property image data generating unit 37 (FIG. 12) according to a third example will be described. In this example, a beta distribution is obtained in the same manner as have been described in the first example, and statistics values to be used are selected according to the distribution shape thereof. For example, in the case where the shape of the beta distribution is J-shaped, variance is used as a parameter. Further, as shown in FIG. 19, in the case where the shape of the beta distribution is U-shaped, the data is divided into two at the broken line in the drawing, and an average value of variances calculated with respect to the regions A and B is used as a parameter.

When the shape is recognized, pattern matching, similarity determination using the least-square method, or similarity determination to theoretical figures of statistics parameters may be performed. In this case, mode, median, rth moment about mean can be used as the statistics parameters.

Either or both of the boundary correction unit (FIG. 9) in the third embodiment and the reflectance correction unit (FIG. 11) in the fourth embodiment may be further provided in the ultrasonic imaging apparatus according to the embodiment.

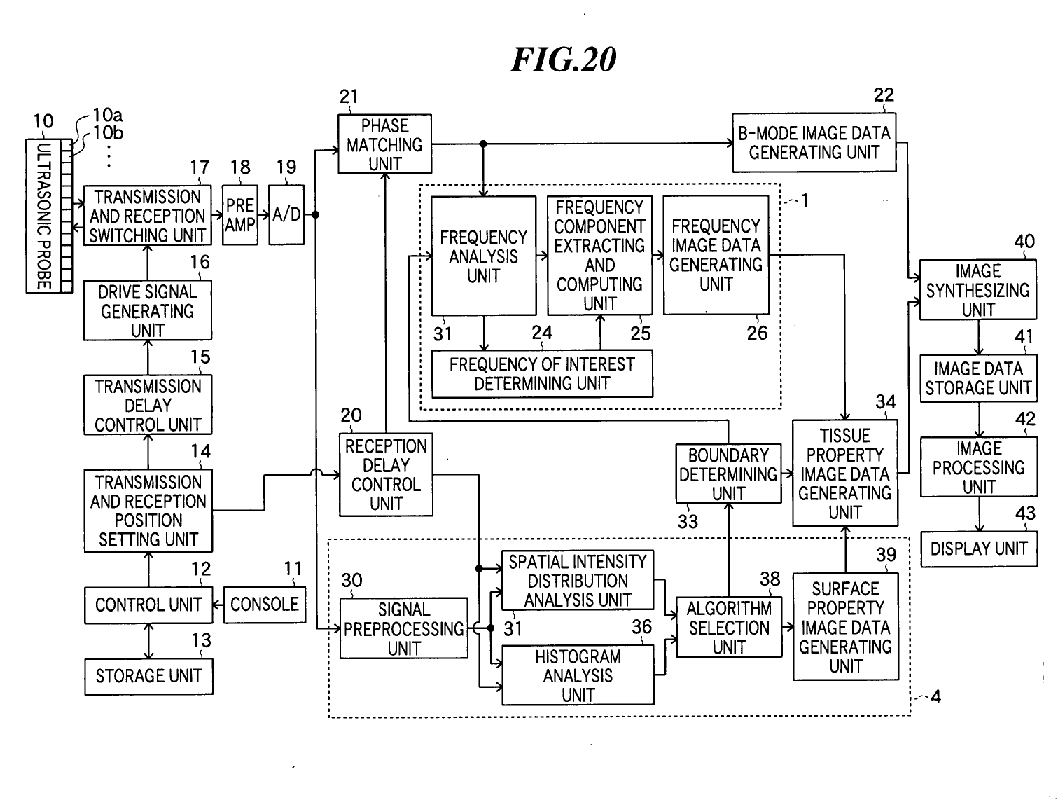

Next, an ultrasonic imaging apparatus according to the sixth embodiment of the present invention will be described. FIG. 20 is a block diagram showing a constitution of the ultrasonic imaging apparatus according to the embodiment. In the ultrasonic imaging apparatus shown in FIG. 20, the image data generating means 4 further has a histogram analysis unit 36 and an algorithm selection unit 38 compared to the image data generating means 2 shown in FIG. 1, and a surface property image data generating unit 39 in place of the surface property image data generating unit 32. The image data generating means 4 is a data processing system for generating surface property image data. Other constitution is the same as that of the ultrasonic imaging apparatus shown in FIG. 1. Further, the operation of the histogram analysis unit 36 is the same as have been described in the fifth embodiment of the present invention.

The algorithm selection unit 38 provides a statistics value to be used for generating surface property image data and an algorithm for surface property image data generation corresponding to the kind of the statistics value from the spatial intensity distribution analysis information generated in the spatial intensity distribution analysis unit 31 and the histogram analysis information generated in the histogram analysis unit 36 to the surface property image data generating unit 39. The surface property image data generating unit 39 generates surface property image data by processing the statistics value using the provided algorithm. The algorithms corresponding to the kinds of the statistics values are the same as those have been described in the first to fifth embodiments of the present invention.

Which of the spatial intensity distribution analysis information and the histogram analysis information is used may be set in advance according to conditions such as the number of reception signals depending on the aperture of the ultrasonic transducer array, the intensity of transmitted ultrasonic beam, etc. Further, the use of a combination of the spatial intensity distribution analysis information and the histogram analysis information may be set in advance according to the kind of statistics value. For example, the histogram analysis information is used for the statistics value (variance or the like) representing a surface property of a reflector and the spatial intensity distribution analysis information is used for the statistics value (kurtosis or the like) representing the inclination of the reflector. Alternatively, the statistics value to be used may be selected by the command of the operator input using the console 11. In this case, the operator may input commands while watching an ultrasonic image displayed on the display unit 33.

Thus, the use of combinations of the spatial intensity distribution analysis information and the histogram analysis information enables display of ultrasonic images more suitable for diagnoses.

Either or both of the boundary correction unit (FIG. 9) in the third embodiment and the reflectance correction unit (FIG. 11) in the fourth embodiment maybe further provided in the ultrasonic imaging apparatus according to the embodiment.

In the first to sixth embodiments of the present invention, when image data representing property of boundaries and image data representing property of the internal region and/or the external region of the boundaries are generated, other publicly known methods may be used. For example, as disclosed in JP-P2001-170046A, using the difference between ultrasonic echoes in two positions along the depth direction, tissue property image data in a region between those positions may be generated. Alternatively, image data representing the internal region and/or the external region of boundaries may be generated using elastic nature of the object that is detected by transmitting and receiving ultrasonic waves while applying pressure to the object. For details on an elastic image expressing such elastic nature of the object, refer to Japanese Patent Publication JP-B-2629734, for example.

1. An ultrasonic imaging apparatus comprising:
an ultrasonic probe including plural ultrasonic transducers for transmitting ultrasonic waves toward an object to be inspected and receiving ultrasonic waves reflected from the object to output plural reception signals;
boundary information generating means for generating information representing positions of boundaries between plural different tissues based on the plural reception signals respectively output from said plural ultrasonic transducers;
first image data generating means for generating image data representing property of a first region and/or a second region divided by the boundaries, based on the plural reception signals;
second image data generating means for generating image data representing property of boundaries based on the plural reception signals; and
tissue property image data generating means for generating image data representing tissue property with respect to a region within the object by locating an image represented by the image data generated by said first image data generating means and an image represented by the image data generated by said second image data generating means in the region, based on the information representing positions of boundaries.

2. An ultrasonic imaging apparatus according to claim 1, wherein said boundary information generating means generates the information representing positions of boundaries by determining positions, where intensity becomes peak in signals obtained by performing phase matching with respect to the plural reception signals and representing sound rays, are boundaries.

3. An ultrasonic imaging apparatus according to claim 1, wherein said boundary information generating means generates the information representing positions of boundaries based on parameters obtained by using an interrelationship among the plural reception signals.

4. An ultrasonic imaging apparatus according to claim 1, wherein said boundary information generating means generates the information representing positions of boundaries by determining whether or not positions, where intensity becomes peak in signals representing sound rays obtained by performing phase matching with respect to the plural reception signals and representing sound rays, are boundaries based on parameters obtained by using an interrelationship among the plural reception signals.

5. An ultrasonic imaging apparatus according to claim 3, wherein said boundary information generating means uses as said interrelationship one of a spatial intensity distribution of the plural reception signals and statistical property among the plural reception signals.

6. An ultrasonic imaging apparatus according to claim 4, wherein said boundary information generating means uses as said interrelationship one of a spatial intensity distribution of the plural reception signals and statistical property among the plural reception signals.

7. An ultrasonic imaging apparatus according to claim 5;, wherein said boundary information generating means obtains the statistical property among the reception signals by utilizing a beta distribution.

8. An ultrasonic imaging apparatus according to claim 6, wherein said boundary information generating means obtains the statistical property among the reception signals by utilizing a beta distribution.

9. An ultrasonic imaging apparatus according to claim 1, wherein said first image data generating means generates the image data representing property of the first region and/or the second region based on frequency characteristics of signals obtained by performing phase matching with respect to the plural reception signals and representing sound rays.

10. An ultrasonic imaging apparatus according to claim 1, wherein said second image data generating means generates the image data representing property of boundaries by obtaining an interrelationship among the plural reception signals and using the interrelationship as a parameter.

11. An ultrasonic imaging apparatus according to claim 10, wherein said second image data generating means uses as said interrelationship one of a spatial intensity distribution of the plural reception signals and statistical property among the plural reception signals.

12. An ultrasonic imaging apparatus according to claim 11, wherein said second image data generating means obtains the statistical property among the reception signals by utilizing a beta distribution.

13. An ultrasonic imaging apparatus according to claim 1, further comprising:
B-mode image data generating means for generating B-mode image data with respect to the region within the object by performing phase matching on the plural reception signals; and
means for generating synthesized image data by superimposing an image represented by the image data generated by said tissue property image data generating means upon an image represented by the B-mode image data.
